(19) United States
(12) Patent Application Publication
(10) Pub. No.: US 2020/0316241 A1
(43) Pub. Date: Oct. 8, 2020

(54) DEODORANT DISPERSION, DEODORANT-CONTAINING PROCESSING SOLUTION, AND METHOD FOR PRODUCING DEODORANT PRODUCT

(71) Applicant: Koji SUGIURA, Nagoya-shi (JP)

(72) Inventor: Koji SUGIURA, Nagoya-shi (JP)

(21) Appl. No.: 16/304,823

(22) PCT Filed: Mar. 9, 2017
(86) PCT No.: PCT/JP2017/009434

§ 371 Date: Nov. 27, 2018

(30) Foreign Application Priority Data
Jun. 8, 2016 (JP) 2016-114521

(51) Int. Cl. A61L 9/012; A61L 9/014

(52) U.S. Cl. CPC A61L 9/012; A61L 9/014; A61L 2209/134; A61L 2209/22; A61L 2209/21

(57) ABSTRACT

The deodorant dispersion of the present invention includes (A) two or more powdery inorganic chemical adsorbents, (B) a dispersing agent, and (C) a dispersion medium, a content proportion of the component (A) is in a range from 10% to 30% by mass based on a total of the deodorant dispersion, a content proportion of the component (B) is in a range from 1% to 10% by mass based on a total of the deodorant dispersion, and a median particle diameter of a dispersoid is in a range from 0.1 μm to 0.4 μm. A gas adsorption capacity of the component (A) is preferably 25 mL/g or more.

### FIELD OF THE INVENTION

The present invention relates to a dispersion containing a deodorant effective for a complex odor containing two or more odor components and to a deodorant containing processing solution for use in producing deodorant products.

### BACKGROUND ART

In recent years, interest in odors in daily life has been increasing, and stationary-type and spray-type deodorant products have been proposed to reduce unpleasant odors or bad odors. Further, various deodorant products have been commercialized, such as wallpapers, curtains, carpets, mats, sofas, filters, and clothes having a deodorizing effect. Examples of unpleasant odors or bad odors include foul odors of raw garbage, odors of human or animal excrement, sweat odors, body odors of old people, aldehyde generated from furniture and household appliances, tobacco odors, and the like.

Activated carbon has heretofore been widely used as a deodorant. However, activated carbon is physically adsorptive, and therefore it is difficult to use activated carbon as a general deodorant for various purposes. The disadvantage of physical adsorptivity is that activated carbon adsorbs any gas component and therefore continues to adsorb gases other than bad odors in the open air so that its adsorptive capacity is immediately saturated. When the amount of adsorption is saturated or the ambient temperature increases, activated carbon discharges gases once adsorbed and therefore also becomes a source of bad odors. Accordingly, activated carbon can be used only for replacement products. Further, activated carbon is black, and therefore has a problem that it is difficult to obtain white or chromatic deodorant products. White or light-colored chemically adsorptive deodorants have been developed.

For example, Patent Literature 1 discloses a deodorant effective for a sulfur-based gas, which consists of a zinc oxide fine particle having a specific surface area of 40 to 100 m²/g, a hydrogen sulfide-deodorizing capacity of 3.0 mmol/g or more, and a primary particle diameter of 0.2 m or smaller. Patent Literature 2 discloses a white deodorant effective for an ammonia-based gas, an amine-based gas, or a sulfur-based gas, which consists of a particle in which zinc oxide and aluminum oxide and/or silicon oxide are closely bonded. Further, Patent Literature 3 discloses an aqueous deodorant dispersion effective for an aldehyde gas or a basic gas, which is characterized by containing: a deodorant in which a compound having a primary amino group is carried on an organic or inorganic carrier; and a deodorant consisting of aluminum silicate.

Further, Patent Literature 4 discloses a deodorant consisting of a layered zirconium phosphate represented by Zr₁₋ₓHfₓHₐ(PO₄)_{b}.nH₂O.

In these chemically-adsorptive deodorants, reaction mechanism of each deodorant is different. Usually, one kind of the deodorant adsorbs only one or two odor components selected from, for example, a basic gas, an acidic gas, a sulfur-based gas, an aldehyde gas, and the like. The above-described unpleasant odors or bad odors usually drift in the air as complex odors containing two or more odor components, and therefore deodorant active ingredients that can respectively deal with odor components need to be combined. Accordingly, plural deodorants are used in combination to deal with a complex odor.

For example, Patent Literature 5 discloses a deodorant composition containing an aldehyde-based gas deodorant in which at least one selected from the group consisting of succinic dihydrazide, carbohydrazide, and oxalic dihydrazide is carried on at least one selected from the group consisting of a silicate compound and a tetravalent metal phosphoric salt, and at least one selected from the group consisting of a sulfur-based gas deodorant, a basic gas deodorant, and an organic acidic gas deodorant; and a deodorant composition containing an aldehyde-based gas deodorant in which at least one selected from the group consisting of succinic dihydrazide, carbohydrazide, and oxalic dihydrazide is carried on a mixture of magnesium silicate and at least one selected from the group consisting of an aluminum silicate, zeolite, and a tetravalent metal phosphoric salt, and at least one selected from the group consisting of a sulfur-based gas deodorant, a basic gas deodorant, and an organic acidic gas deodorant. A suspension containing the deodorant composition and a binder is also described in it.

Patent Literature 6 discloses an adsorptive composition effective for an acidic gas and a basic gas, which contains silicon dioxide, a tetravalent metal water-insoluble phosphoric salt, and a bivalent metal hydroxide. Further, Patent Literature 7 discloses a deodorant system having: a solution A containing (a) smectite, (b) aluminum silicate, (c) a binder resin, (d) a modified silicone compound, and (c1) water for solution A; and a solution B containing (f) zinc oxide and (c2) water for solution B.

### PRIOR ART LITERATURE

### Patent Literature

Patent Literature 1: JP-A 2003-52800
Patent Literature 2: JP-A S63-246167
Patent Literature 3: JP-A 2000-279500
Patent Literature 4: JP-A 2012-254925
Patent Literature 5: WO 2004/058311
Patent Literature 6: JP-A H7-813
Patent Literature 7: JP-A 2015-171449

### SUMMARY OF THE INVENTION

### Problems that the Invention is to Solve

In recent years, a deodorant containing processing solution containing a deodorant and a binder has been used to impart a deodorizing function to a fiber sheet, paper, and the like. Since various binders are used depending on the intended use of a deodorant product, a deodorant containing dispersion is used as a raw material for production of a deodorant containing processing solution. As described above, two or more deodorants are used to deal with a complex odor. However, a dispersion containing two or more deodorants has a problem that its storage stability is insufficient because the deodorants precipitate to form a cake and do not again disperse after storage for a certain period of time. An object of the present invention to provide a dispersion that includes a deodorant effective for a complex odor containing two or more odor components and has excellent storage stability; a deodorant containing processing solution that can lead to a deodorant product having excellent washing durability; and a method for producing a deodorant product.

### Means for Solving the Problems

The present inventor has found that a deodorant dispersion containing an inorganic chemical adsorbent powder and a dispersing agent at a specific ratio has excellent storage stability, and is therefore suitable as a raw material of a deodorant containing processing solution for use in obtaining a deodorant product effective for a complex odor. Further, the present inventor has also found that a deodorant product obtained using this deodorant containing processing solution has excellent washing durability.

The present invention is as follows.

1. A deodorant dispersion comprising (A) two or more powdery inorganic chemical adsorbents, (B) a dispersing agent, and (C) a dispersion medium, characterized in that:

a content proportion of the component (A) is in a range from 10% to 30% by mass based on a total of the deodorant dispersion;

a content proportion of the component (B) is in a range from 1% to 10% by mass based on a total of the deodorant dispersion; and

a median particle diameter of a dispersoid is in a range from 0.1 m to 0.4 m.

2. The deodorant dispersion according to 1 above,

wherein a gas adsorption capacity of the component (A) is 25 mL/g or more.

3. The deodorant dispersion according to 1 or 2 above,

wherein at least one of the inorganic chemical adsorbents constituting the component (A) is an amorphous substance.

4. The deodorant dispersion according to any one of 1 to 3 above,

wherein the component (A) comprises a compound represented by a general formula (1):

aM¹₂O.bM²O.cM³₂O₃.dM⁴O₂.eP₂-O₅.fH₂O (1)

wherein M¹ represents an alkali metal atom, M² represents a bivalent metal atom, M³ represents a trivalent metal atom, M⁴ represents a tetravalent metal atom, at least one of a, b, c, d and e is a positive number and a rest is 0 or a positive number, and f is a positive number.

5. The deodorant dispersion according to 4 above,

wherein M¹₂O in the general formula (1) is Na₂O.

6. The deodorant dispersion according to 4 above,

wherein M²O in the general formula (1) is MgO, ZnO or CuO.

7. The deodorant dispersion according to 4 above,

wherein M³₂O₃ in the general formula (1) is Al₂O₃.

8. The deodorant dispersion according to 4 above,

wherein M⁴O₂ in the general formula (1) is SiO₂, TiO₂ or ZrO₂.

9. A deodorant containing processing solution characterized by comprising the deodorant dispersion according to any one of 1 to 8 above, and an adhesive agent.
10. A production method of a deodorant product characterized by comprising a process for applying the deodorant containing processing solution according to 9 above on a base material, and a process for drying a coated article.

### Effects of the Invention

The deodorant dispersion of the present invention is excellent in storage stability. Therefore, the deodorant dispersion is useful as a raw material for a deodorant containing processing solution when a deodorant product is produced.

Further, when the deodorant containing processing solution of the present invention is used, a deodorant product having excellent washing durability can be efficiently produced.

### EMBODIMENT FOR CARRYING OUT THE INVENTION

Hereinafter, the present invention will be described in detail.

The deodorant dispersion of the present invention consists of a one liquid containing (A) two or more powdery inorganic chemical adsorbents, (B) a dispersing agent, and (C) a dispersion.

The component (A) consists of two or more powdery inorganic chemical adsorbents. Usually, each of the inorganic chemical adsorbents adsorbs only one or two odor components selected from, for example, a basic gas, an acidic gas, a sulfur-based gas, an aldehyde gas, and the like. Therefore, in the present invention, two or more powdery inorganic chemical adsorbents are used in combination to get rid of a complex odor.

The gas adsorption capacity per gram of all the powdery inorganic chemical adsorbents is preferably 10 mL or more, and more preferably 25 mL or more. In the present invention, the gas adsorption capacities of the two or more inorganic chemical adsorbents may be the same or different from each other. It is to be noted that the gas adsorption capacity refers to the maximum volume of an odor component that can be absorbed or adsorbed by the adsorbent, and is measured in the following manner.

The adsorbent is placed in a test bag made of a vinyl alcohol-based polymer or a polyester that is hard to adsorb odor components and is not air permeable, and the test bag is sealed. Subsequently, an odor gas is introduced into the sealed test bag. Then, immediately after the introduction of an odor gas and after the elapse of a certain period of time, the concentration of the odor gas remaining in the test bag is measured. After that, a time at which the residual gas concentration after the elapse of a certain period of time is 1/10 or more of the initial gas concentration is determined to be a breakpoint with respect to deodorizing performance. Using a difference between the residual gas concentration at the time and the total sum of the initial gas concentrations, an adsorption capacity of the gas absorbed or adsorbed by the adsorbent is determined.

In the present invention, the inorganic chemical adsorbent is not particularly limited. The inorganic chemical adsorbents preferably contain an amorphous substance whose deodorizing performance is less likely to be reduced even when combined. It is noted that the amorphous substance is a substance having an X-ray diffraction image obtained by powder X-ray diffraction measurement in which no peak is observed, or a low crystalline substance having an X-ray diffraction image obtained by powder X-ray diffraction measurement in which a peak having a half-width of 0.5 degrees or more is observed. The component (A) may be of a powder in which all the inorganic chemical adsorbents are amorphous, or of a powder in which a part of the inorganic chemical adsorbents is crystalline.

The inorganic chemical adsorbent is preferably of an oxide containing at least one metal atom selected from an alkali metal atom, a bivalent metal atom, a trivalent metal atom, and a tetravalent metal atom, or of a composite containing the same. For example, a compound represented by the following general formula (1) is preferred.

aM¹₂O.bM²O.cM³₂O₃.dM⁴O₂.eP₂O₅.fH₂O (1)

(In the formula, M¹ is an alkali metal atom, M² is a bivalent metal atom, M³ is a trivalent metal atom, M⁴ is a tetravalent metal atom, at least one of a, b, c, d, and e is a positive number, the rest is 0 or a positive number, and f is a positive number.)

In the general formula (1), M¹ is an alkali metal atom, and examples thereof include Li, Na, K, Rb, Cs, and the like. Among them, Na is preferred. M² is a bivalent metal atom, and examples thereof include an alkaline earth metal atom such as Be, Mg, and Ca; Ba, Cu, Zn, Fe, Co, Ni, Ru, Pd, Pt, Pb, Cd, and the like. Among them, Cu, Mg and Zn are preferred. M³ is a trivalent metal atom, and examples thereof include Al, Fe, and the like. Among them, Al is preferred. M⁴ is a tetravalent metal atom, and examples thereof include Si, Ti, Zr, and the like. Among them, Si is preferred.

Each of M¹, M², M³, and M⁴ constituting the general formula (1) may be of only one atom or two or more atoms.

Specific examples of the compound represented by the general formula (1) are represented by formulae (1-1) to (1-16). In the following, a, b, c, d, and e are each 0 or a positive number, f is a positive number, b=b₁+b₂, c=c+c₂, and d=d₁+d₂.

aM¹₂O.dSiO₂.fH₂O (1-1)

aM¹₂O.bM²O.dSiO₂.fH₂O (1-2)

aM¹₂O.cM³₂O₃.dSiO₂.fH₂O (1-3)

aM¹₂O.d₁M⁴O₂.d₂SiO₂.fH₂O (1-4)

aM¹₂O.bM²O.eP₂O₅.fH₂O (1-5)

aM¹₂.cM³₂O₃.eP₂O₅.fH₂O (1-6)

aM¹₂O.dM⁴O.eP₂O₅.fH₂O (1-7)

aM¹₂O.bM²O.cAl₂O₃.fH₂O (1-8)

aM¹₂.c₁M³₂O₃.c₂Al₂O₃.fH₂O (1-9)

aM¹₂O.dM⁴O₂.cA₂O₃.fH₂O (1-10)

aM¹₂O.bZnO.fH₂O (1-11)

cM³₂O₃.bZnO.fH₂O (1-12)

aM¹₂O.b₁M²O.b₂ZnO.fH₂O (1-13)

aM¹₂O.cM³₂O₃.bZnO.fH₂O (1-14)

aM¹₂O.dZrO₂.fH₂O (1-15)

aM¹₂O.dTiO₂.fH₂O (1-16)

The shape and particle diameter of the inorganic chemical adsorbent powder is not particularly limited. The BET specific surface area is preferably 30 m²/g or more, and more preferably in a range from 50 to 400 m²/g. An amorphous inorganic chemical adsorbent powder having a specific surface area of 30 m²/g or more has a large gas adsorption capacity, and even when the powders come into contact with each other in the dispersion medium, only reaction groups on the powder particle surfaces influence each other. Therefore, it is considered that even when the amorphous inorganic chemical adsorbent powder is blended, its deodorizing effect is hardly reduced because reaction groups inside the powder are hardly influenced and deodorizing performance is less likely to be cancelled out.

The color of the inorganic chemical adsorbent powder is not particularly limited, but white to light color is preferred. In this case, the color of the powder preferably has L value of 92 to 98, a value of −1 to −6, and b value of 4 to 10, and more preferably has L value of 94 to 97, a value of −1.5 to 5, and b value of 5 to 9 in the Lab color space.

From a viewpoint of storage stability, a content proportion of the component (A) in the deodorant dispersion of the present invention is in a range from 10% to 30% by mass, preferably from 10% to 27% by mass, and more preferably from 12% to 25% by mass with respect to a total of the dispersion.

The dispersing agent (B) is not particularly limited. In a case where the dispersion medium (C) is an aqueous dispersion medium, a polycarboxylic acid-based dispersing agent, a naphthalenesulfonic acid-formalin condensate-based dispersing agent, polyethylene glycol, an alkylsulfonic acid-based dispersing agent, a quaternary ammonium-based dispersing agent, a higher alcohol alkylene oxide-based dispersing agent, a polyphosphoric acid-based dispersing agent, and the like may be used. Among them, a polycarboxylic acid-based dispersing agent and a higher alcohol alkylene oxide-based dispersing agent are preferred. In a case where the dispersion medium (C) is an organic solvent, a polycarboxylic acid alkyl ester-based dispersing agent, a polyether-based dispersing agent, a polyalkylaminc-based dispersing agent, a polyhydric alcohol cster-based dispersing agent, an alkyl polyamine-based dispersing agent, and the like may be used.

The solubility of the component (B) in the dispersion medium (C) is not particularly limited. In the present invention, the component (B) is preferably soluble in the dispersion medium (C).

From a viewpoint of storage stability, a content proportion of the dispersing agent (B) in the deodorant dispersion of the present invention is in a range from 1% to 10% by mass, and preferably from 3% to 9% by mass with respect to a total of the dispersion.

The dispersion medium (C) contained in the deodorant dispersion of the present invention may be water, an alcohol, a ketone, an ester, a hydrocarbon, and the like. Among them, water is preferred.

The deodorant dispersion of the present invention contains the above-described components (A), (B) and (C), and the inorganic chemical adsorbent powders constituting the component (A) form a dispersoid independently or by agglomerating. When the component (A) agglomerates, the component (B) sometimes intermediates. The median particle diameter of the dispersoid contained in the deodorant dispersion of the present invention is in a range from 0.1 to 0.4 m, and preferably from 0.15 to 0.35 m from a viewpoint of storage stability. The median diameter can be measured with a laser diffraction particle size distribution analyzer. An upper limit of the particle diameter of the dispersoid is usually 5 m, and preferably 3 m from a viewpoint of storage stability and processability when the dispersing agent is used.

A method for producing the deodorant dispersion of the present invention is not limited in particular. Preferred production methods are as follows.

(1) A method in which a raw material for two or more powdery inorganic chemical adsorbents, a dispersing agent, and a dispersion medium are mixed and subjected to wet pulverization.
(2) A method in which a raw material for one powdery inorganic chemical adsorbent, a dispersing agent, and a dispersion medium are mixed and subjected to wet pulverization to prepare a first dispersion; a raw material for another powdery inorganic chemical adsorbent, a dispersing agent, and a dispersion medium are mixed and subjected to wet pulverization to prepare an n-th dispersion (n≥2); and then the first dispersion and the n-th dispersion are mixed.

When the wet pulverization is performed, a pulverizer such as a ball mill, a bead mill, a jet mill, and a mixing mill may be used.

In the methods (1) and (2), a dispersion medium may be added, if necessary, after the wet pulverization.

The viscosity of the deodorant dispersion of the present invention depends on types, contents, and the like of the component (A). The viscosity of the deodorant dispersion as measured with a B-type viscometer is preferably 1,200 cPs or less, and more preferably 800 cPs or less. A deodorant dispersion having a viscosity measured with a B-type viscometer of 1200 cPs is suitable as a raw material for a deodorant containing processing solution or the like that will be described later.

The deodorant containing processing solution of the present invention contains the above-described deodorant dispersion of the present invention and an adhesive agent. That is, the deodorant containing processing solution of the present invention contains two or more powdery inorganic chemical adsorbents, a dispersing agent, a dispersion medium, and an adhesive agent.

The adhesive agent is not limited in particular. A natural resin, a semi-synthetic resin, or a synthetic resin may be used. The solubility of the adhesive agent in the dispersion medium is not particularly limited. Preferred examples of the resin include a novolac type or resol type phenolic resin, an alkyd resin, an aminoalkyd resin, an acrylic resin, a vinyl chloride resin, a vinylidene chloride resin, a silicone resin, a fluororesin, an epoxy resin, a urethane resin, a saturated polyester resin, a melamine resin, and the like. Among them, an acrylic resin, a vinyl chloride resin, a silicone resin, and a urethane resin are particularly preferred. The adhesive agent containing a silicone resin leads to a deodorant product having excellent washing resistance. The silicone resin can be used in combination with other resin.

The content of the adhesive agent in the deodorant containing processing solution of the present invention is not particularly limited. When a total amount of the powdery inorganic chemical adsorbents is taken as 100 parts by mass, the content of the adhesive agent is preferably in a range from 30 to 500 parts by mass, and more preferably from 50 to 300 parts by mass, because excellent adhesion to a base material that will be described later can be achieved while the powdery inorganic chemical adsorbents are exposed.

When a total amount of the powdery inorganic chemical adsorbents, the dispersing agent, and the adhesive agent is taken as 100% by mass, the content proportion of the powdery inorganic chemical adsorbents, the content of the dispersing agent, and the content of the adhesive in the deodorant containing processing solution of the present invention are respectively preferably from 9% to 70% by mass, from 1% to 20% by mass, and from 10% to 90% by mass, and more preferably from 20% to 50% by mass, from 1% to 20% by mass, and from 20% to 60% by mass.

The deodorant containing processing solution of the present invention can be produced by mixing the above-described deodorant dispersion of the present invention and a raw material for an adhesive agent. Alternatively, a mixture obtained by mixing one deodorant dispersion and a raw material for an adhesive agent may be mixed with other deodorant dispersion or with a mixture of other deodorant dispersion and a raw material for an adhesive agent. The raw material for adhesive agent may be a polymer dispersion such as an emulsion and a latex, or a polymer solution obtained by dissolving an adhesive agent in a medium. In either case, it is preferred that the medium of the deodorant dispersion and the medium of the raw material for adhesive agent arc the same or have an affinity for each other.

Various deodorant products can be produced using the deodorant containing processing solution of the present invention. The production method for a deodorant product of the present invention includes a process for applying the deodorant containing processing solution on a base material (hereinafter, referred to as “first process”), and a process for drying a coated article (hereinafter, referred to as “second process”).

The base material is not particularly limited, and may be, for example, a fiber, a film, a sheet, a formed material, or an unformed material. The constituent material of the base material is not limited, either. The material may be either an organic material or an inorganic material or may be a combination thereof.

The fiber may be either a natural fiber or a synthetic fiber. Further, the fiber may be a short fiber, a long fiber, a composite fiber having a core-sheath structure, or the like.

As the film, a thermoplastic resin film or a cured resin film may be used.

Examples of the sheet include: a fiber sheet (including paper) containing two or more of the above-described fibers, such as a knitted fabric, a woven fabric, and a nonwoven fabric; a laminate of films; and a laminate of a fiber sheet and a film.

In the first process, a method for applying the deodorant containing processing solution on a base material is appropriately selected depending on a type, shape, and the like of the base material. Examples of the method include dip coating, spray coating, roll coating, gravure coating, knife coating, kiss coating, and the like. Further, the deodorant containing processing solution is used selecting appropriately the composition, liquid property, and the like depending on a type of the base material. For example, when the deodorant containing processing solution contains water as a medium, its preferred pH is in a range from 6 to 9.

An amount of the deodorant containing processing solution to be applied is not particularly limited. When the applying amount of the deodorant containing processing solution is increased, a deodorizing effect can be more strongly exerted and maintained for a longer period of time in general. But the deodorizing effect is not increased in proportion to the applied amount of the deodorant when the deodorant amount is a certain level or more. Therefore, the amount of the deodorant containing processing solution applied is preferably in a range from 0.1 to 20 g in terms of the solid weight of the deodorant per m² of the base material.

After that, the coated article is dried by, for example, heating in the second step. The heating temperature is preferably 80° C. or higher, and more preferably in a range from 85° C. to 130° C.

A deodorant sheet obtained by using a fiber sheet as the base material and applying the deodorant containing processing solution of the present invention on the sheet may be a sheet in which the deodorant may be contained in the entire base material from one side to the other side thereof, a sheet in which the deodorant is provided in a surface layer on one side or the other side thereof, or a sheet in which the deodorant is provided in the inside thereof except for surface layers.

When a deodorant sheet is produced using a paper as the base material, the deodorant containing processing solution may be introduced into the paper in a paper-making process.

The deodorant sheet obtained by the method of the present invention can be used as, for example, a medical packing paper, a food packing paper, a packing paper for electrical devices, a nursing paper product, a freshness-keeping paper, a paper cloth, an air cleaning filter, a wall paper, a tissue paper, a toilet paper, and the like.

Further, a deodorant fiber obtained by using a fiber as the base material and applying the deodorant containing processing solution of the present invention on it can be used for, for example, producing a fiber product including clothes such as underwear, socks, and aprons; nursing clothes; futons; cushions; blankets; carpets; sofas; air filters; futon covers; curtains; car sheets; and the like.

The deodorant containing processing solution of the present invention can be used for forming a film on a surface of a resin molded article or a foamed molded article.

The resin molded articles or foamed molded articles having a deodorant-containing film can be used as for example, home appliances such as air cleaners and refrigerators; general household products such as garbage boxes and drainers; nursing care products such as portable toilets; and the like.

### EXAMPLES

Hereinafter, the present invention will be described in more detail with reference to Examples, but is not limited to these Examples. It is to be noted that “%” refers to % by mass.

### 1. Production and Evaluation of Deodorant Dispersions

Deodorant dispersions were produced using the following raw materials.

### 1-1. Inorganic Chemical Adsorbent Powders

Inorganic chemical adsorbent powders shown in Table 1 were used.

TABLE 1

| | | Specific | Gas adsorption | |
|---|---|---|---|---|
| | | surface | capacity | |
| | | area | Compound | |
| Inorganic chemical adsorbent powder | | (m²/g) | Name | (mL/g) |
| Amorphous sodium aluminum silicate | | 220 | Ammonia | 40 |
| Amorphous aluminum silicate | A | 250 | Ammonia | 40 |
| | B | 220 | Ammonia | 40 |
| | C | 240 | Ammonia | 40 |
| Amorphous magnesium silicate | | 130 | Ammonia | 25 |
| Amorphous aluminum phosphate | | 130 | Ammonia | 70 |
| Amorphous aluminum zinc oxide | A | 80 | Hydrogen | 85 |
| | | | sulfide | |
| | | | Acetic acid | 33 |
| | B | 90 | Hydrogen | 85 |
| | | | sulfide | |
| | | | Acetic acid | 32 |
| Amorphous magnesium aluminate | | 90 | Acetic acid | 31 |
| Amorphous zirconium hydroxide | | 35 | Acetic acid | 28 |
| Amorphous copper silicate | | 280 | Acetic acid | 25 |
| | | | Hydrogen | 85 |
| | | | sulfide | |
| Amorphous silica | | 330 | Ammonia | 25 |
| Amorphous titanium oxide | A | 90 | Ammonia | |
| | | | Acetic acid | 27 |
| | B | 70 | Ammonia | 70 |
| | | | Acetic acid | 5 |

| | | | |
|---|---|---|---|
| Crystalline titanium oxide | 6 | Acetic acid | 5 |
| Crystalline aluminum silicate | 240 | Ammonia | 20 |
| Crystalline aluminum zinc oxide | 11 | Acetic acid | 5 |
| | | Hydrogen | 15 |
| | | sulfide | |
| Crystalline titanium phosphate | 15 | Ammonia | 155 |

### 1-2. Dispersing Agent

### (1) Polycarboxylic Acid-Based Dispersing Agent

“Nopcosperse 44-C” (trade name)” manufactured by San Nopco was used.

### (2) Higher Alcohol Alkylene Oxide-Based Dispersing Agent

“NOIGEN LF-202N” (trade name)” manufactured by DKS Co. Ltd. was used.

### 1-3. Dispersion Medium

Water was used.

### Examples 1-1 to 1-6 and Comparative Examples 1-1 to 1-9

The inorganic chemical adsorbent powder(s), the dispersing agent, and water were mixed based on formulations shown in Tables 2 and 3, and the mixtures were subjected to wet pulverization using a bead mill to obtain deodorant dispersions (D1) to (D15). The color, median diameter, viscosity, and storage stability of the obtained deodorant dispersions were evaluated by the following methods. The results are shown in Tables 2 and 3.

### (1) Specific Surface Area of Inorganic Chemical Adsorbent Powder

The surface area per gram of the inorganic chemical adsorbent powder was measured with a BET specific surface area meter.

### (2) Gas Adsorption Capacity of Inorganic Chemical Adsorbent Powder

At about 20° C., the inorganic chemical adsorbent powder was placed in a test bag (inner volume: 4 L) made of a vinyl alcohol-based polymer, the test bag was sealed, and 3 L of an odor gas (ammonia gas, acetic acid gas, or hydrogen sulfide gas) of 100 to 1000 ppm was introduced into the sealed test bag. Then, immediately after the introduction of an odor gas and after two hours, the concentration of the odor gas remaining in the test bag was measured. A time at which the residual gas concentration was 1/10 or more of the initial gas concentration was determined to be a breakpoint with respect to deodorizing performance, and then the difference between the residual gas concentration at this time and the total sum of the initial gas concentrations was used to determine the total volume of the gas adsorbed by the adsorbent powder. The total adsorbed gas volume (mL) was divided by the amount (g) of the inorganic chemical adsorbent powder used in the test to determine a gas adsorption capacity (mL/g).

### (3) Median Particle Diameter of Dispersoid in Dispersion

The dispersion as a sample was subjected to a measurement with a laser diffraction particle size distribution analyzer “MS 2000” (model name) manufactured by Malvern Instruments Ltd., and the median particle diameter D50 of a dispersoid contained in the deodorant dispersion was analyzed on a volume basis.

### (4) Viscosity of Deodorant Dispersion

B-type viscometer was used to measure the viscosity of the deodorant dispersion at a temperature of about 20° C.

### (5) Storage Stability of Deodorant Dispersion

One liter of the deodorant dispersion placed in a polyethylene bottle was stored in a thermostat at a temperature of 50° C. for 1 month. After the storage, the polyethylene bottle was shaken by hand to evaluate storage stability according to the following criteria: “◯” redispersibility was excellent and viscosity was increased less than two times; and “x” viscosity was increased two times or more or the deodorant dispersion was not redispersed.

TABLE 2

| | | | | | | | Deodorant dispersion | | | | |
|---|---|---|---|---|---|---|---|---|---|---|---|
| | | | | | | | | | Median | | |
| | | | | | | | | | particle | Viscos- | Storage |
| | | Inorganic chemical adsorbent | | Dispersing agent | | Water | | | diameter | ity | stabil- |
| | | Type | Parts | Type | Parts | Parts | Type | Color | (μm) | (cPs) | ity |
| Example | 1-1 | Amorphous sodium aluminum silicate | 10 | Polycarboxylic | 5 | 75 | D1 | White | 0.21 | 330 | ∘ |
| | | Amorphous titanium oxide | 5 | acid-based | | | | | | | |
| | | Amorphous aluminum zinc oxide | 5 | | | | | | | | |
| | 1-2 | Amorphous magnesium silicate | 16 | Higher alcohol | 5 | 60 | D2 | White | 0.25 | 880 | ∘ |
| | | Amorphous zirconium hydroxide | 8 | alkylene | | | | | | | |
| | | Amorphous aluminum zinc oxide | 4 | oxide-based | | | | | | | |
| | 1-3 | Amorphous aluminum silicate | 8 | Higher alcohol | 7 | 69 | D3 | Light | 0.33 | 600 | ∘ |
| | | Amorphous aluminum zinc oxide | 16 | alkylene | | | | yellow | | | |
| | | | | oxide-based | | | | | | | |
| | 1-4 | Amorphous aluminum phosphate | 8 | Higher alcohol | 8 | 68 | D4 | Light | 0.19 | 580 | ∘ |
| | | Amorphous copper silicate | 16 | alkylene | | | | blue | | | |
| | | | | oxide-based | | | | | | | |
| | 1-5 | Amorphous aluminum silicate | 8 | Higher alcohol | 8 | 68 | D5 | White | 0.24 | 680 | ∘ |
| | | Amorphous magnesium aluminate | 8 | alkylene | | | | | | | |
| | | Amorphous aluminum zinc oxide | 8 | oxide-based | | | | | | | |
| | 1-6 | Amorphous silica | 8 | Higher alcohol | 5 | 71 | D6 | Light | 0.30 | 710 | ∘ |
| | | Amorphous aluminum zinc oxide | 16 | alkylene | | | | yellow | | | |
| | | | | oxide-based | | | | | | | |

TABLE 3

| | | | | | | | Deodorant dispersion | | | | |
|---|---|---|---|---|---|---|---|---|---|---|---|
| | | | | | | | | | Median | | |
| | | | | | | | | | particle | Viscos- | Storage |
| | | Inorganic chemical adsorbent | | Dispersing agent | | Water | | | diameter | ity | stabil- |
| | | Type | Parts | Type | Parts | Parts | Type | Color | (μm) | (cPs) | ity |
| Comparative | 1-1 | Amorphous aluminum silicate | 4 | Higher alcohol | 2 | 90 | D7 | White | 0.19 | 250 | x |
| Example | | Amorphous aluminum zinc oxide | 4 | alkylene | | | | | | | |
| | | | | oxide-based | | | | | | | |
| | 1-2 | Amorphous aluminum silicate | 8 | Higher alcohol | 4 | 72 | D8 | White | 0.80 | 310 | x |
| | | Crystalline titanium oxide | 16 | alkylene | | | | | | | |
| | | | | oxide-based | | | | | | | |
| | 1-3 | Crystalline aluminum silicate | 8 | Higher alcohol | 4 | 72 | D9 | White | 1.20 | 450 | x |
| | | Crystalline aluminum zinc oxide | 16 | alkylene | | | | | | | |
| | | | | oxide-based | | | | | | | |
| | 1-4 | Amorphous titanium oxide | 24 | Higher alcohol | 4 | 72 | D10 | White | 0.90 | 230 | ∘ |
| | | | | alkylene | | | | | | | |
| | | | | oxide-based | | | | | | | |
| | 1-5 | Amorphous aluminum silicate | 20 | Higher alcohol | 8 | 57 | D11 | White | 0.06 | 2200 | x |
| | | Amorphous aluminum zinc oxide | 15 | alkylene | | | | | | | |
| | | | | oxide-based | | | | | | | |
| | 1-6 | Amorphous magnesium silicate | 8 | None | 0 | 76 | D12 | White | 0.55 | 330 | x |
| | | Amorphous zirconium hydroxide | 16 | | | | | | | | |
| | 1-7 | Amorphous magnesium silicate | 12 | Higher alcohol | 10 | 54 | D13 | White | 0.06 | 2200 | x |
| | | Amorphous zirconium hydroxide | 24 | alkylene | | | | | | | |
| | | | | oxide-based | | | | | | | |
| | 1-8 | Amorphous magnesium silicate | 8 | Polycarboxylic | 4 | 72 | D14 | White | 2.30 | 210 | x |
| | | Amorphous zirconium hydroxide | 16 | acid-based | | | | | | | |
| | 1-9 | Crystalline titanium phosphate | 20 | Polycarboxylic | 4 | 76 | D15 | White | 0.60 | 180 | x |
| | | | | acid-based | | | | | | | |

As is apparent from Table 2 and Table 3, when the deodorant dispersion has an inorganic chemical adsorbent content of a range from 10% to 30% and a median particle diameter of the dispersoid of a range from 0.1 m to 0.4 μm, excellent storage stability is obtained.

### 2. Production and Evaluation of Deodorant Containing Processing Solutions and Deodorant Products

### Examples 2-1 to 2-6 and Comparative Examples 2-1 to 2-10

The deodorant dispersions (D1) to (D15) obtained in Examples 1-1 to 1-6 and Comparative Examples 1-1 to 1-9, the following emulsion binders (E1) to (E4), and water were mixed to prepare deodorant containing processing solutions whose inorganic chemical adsorbent concentration was 2% (see Table 4). Subsequently, each of the deodorant containing processing solutions was applied to a polyester cloth having a basis weight of 200 g/m² such that the deodorant was spread in an amount of 2 g/m², and the coated cloth was dried (105° C.) to obtain a polyester deodorant cloth. Then, the thus obtained polyester deodorant cloth was subjected to the following deodorant test and washing test. In Comparative Example 2-10, the deodorant test and the washing test were performed on the polyester cloth before the spreading process. These results are shown in Table 4.

### <Emulsion Binders>

(E1) Silicone emulsion “KM-2002L-1” (trade name) manufactured by Shin-Etsu Chemical Co., Ltd.
(E2) Acrylic emulsion “NK Binder R-5NH” (trade name) manufactured by Shin Nakamura Chemical Co., Ltd.
(E3) Urethane emulsion “Newcoat UR-300Z” (trade name) manufactured by Shin Nakamura Chemical Co., Ltd.
(E4) A mixture of (E1) and (E3) in a mass ratio of 1:2

### (1) Deodorant Test

The polyester deodorant cloth (10 cm×10 cm) was placed in a test bag made of a vinyl alcohol-based polymer film, 3 L of air containing 30 ppm of acetic acid, 100 ppm of ammonia, and 8 ppm of hydrogen sulfide was introduced into the test bag, and the test bag was sealed. After 2 hours, the concentration of each of the gases was measured using a gas detector tube. At the same time, a blank test was performed in the same manner as described above except that the polyester deodorant cloth was not placed in the test bag. A deodorant level was calculated by taking the concentration of the residual gas of the blank test as 100%.

### (2) Washing Test

Water was placed in a washing tub of a washing machine, and the temperature of the water was set to 40° C. Then, a nonionic detergent was added in an amount of 0.54 g per liter of water to prepare a solution for washing. Subsequently, the polyester deodorant cloth or the polyester cloth and a dummy cloth (cotton towel) were put into the washing tub at a bath ratio of 1:30. The size of each of the cloths is about 1000 cm². Washing was conducted for 5 minutes, and the cloths were dehydrated with a dehydrator. Then, the cloths were rinsed with new water at ordinary temperature for 2 minutes at the same bath ratio. This process was regarded as one washing, and repeated 10 times. After that, the deodorant test described in (1) above was performed.

TABLE 4

| | | | | | | Deodorant | | | | | | | |
|---|---|---|---|---|---|---|---|---|---|---|---|---|---|
| | | | | | | containing | | | | | | | |
| | | Deodorant | | | | processing | Drying | Deodorant level | | | After 10 washings | | |
| | | dispersion | | Emulsion | | solution | temperature | | Acetic | Hydrogen | | Acetic | Hydrogen |
| | | Type | Parts *¹ | Type | Parts *² | Type | (° C.) | Ammonia | acid | sulfide | Ammonia | acid | sulfide |
| Example | 2-1 | D1 | 100 | E4 | 20 | P1 | 100 | 100 | 100 | 100 | 100 | 100 | 91 |
| | 2-2 | D2 | 100 | E4 | 35 | P2 | 100 | 100 | 100 | 100 | 89 | 100 | 91 |
| | 2-3 | D3 | 100 | E1 | 10 | P3 | 100 | 100 | 100 | 100 | 100 | 100 | 100 |
| | 2-4 | D4 | 100 | E4 | 10 | P4 | 100 | 100 | 100 | 100 | 91 | 100 | 100 |
| | 2-5 | D5 | 100 | E1 | 15 | P5 | 100 | 100 | 100 | 100 | 80 | 92 | 91 |
| | 2-6 | D6 | 100 | E2 | 20 | P6 | 100 | 100 | 100 | 100 | 80 | 100 | 81 |
| Comparative | 2-1 | D7 | 100 | E3 | 10 | P7 | Air | 100 | 100 | 100 | 56 | 62 | 61 |
| Example | | | | | | | drying | | | | | | |
| | 2-2 | D8 | 100 | E3 | 20 | P8 | 100 | 100 | 58 | 20 | 53 | 40 | 11 |
| | 2-3 | D9 | 100 | E3 | 20 | P9 | 100 | 100 | 65 | 35 | 53 | 55 | 18 |
| | 2-4 | D10 | 100 | E3 | 25 | P10 | 50 | 89 | 35 | 10 | 65 | 32 | 0 |
| | 2-5 | D11 | 100 | E3 | 30 | P11 | 100 | 100 | 100 | 100 | 67 | 58 | 47 |
| | 2-6 | D12 | 100 | E3 | 10 | P12 | 100 | 100 | 100 | 20 | 55 | 45 | 11 |
| | 2-7 | D13 | 100 | E4 | 10 | P13 | 100 | 100 | 100 | 20 | 72 | 69 | 11 |
| | 2-8 | D14 | 100 | E3 | 10 | P14 | 100 | 100 | 100 | 20 | 35 | 35 | 5 |
| | 2-9 | D15 | 100 | E4 | 10 | P15 | Air | 100 | 5 | 0 | 22 | 5 | 0 |
| | | | | | | | drying | | | | | | |
| | 2-10 | — | — | — | — | — | — | 32 | 35 | 5 | 24 | 30 | 0 |

*¹ An amount of inorganic chemical adsorbent.
*² A solid content of resin.

As is apparent from Table 4, the deodorant towels obtained using the deodorant containing processing solutions of Examples 2-1 to 2-6 had a high deodorizing effect on a complex odor of ammonia, acetic acid, and hydrogen sulfide, and a sufficient deodorizing effect of 80% or more was obtained even after 10 washings. On the other hand, in Comparative Examples 2-1 to 2-9, a deodorizing effect was significantly reduced by washing.

### INDUSTRIAL APPLICABILITY

The deodorant dispersion of the present invention is suitable as a raw material of a deodorant containing processing solution for use in obtaining a deodorant product effective for a complex odor. Further, the method for producing a deodorant product of the present invention is suitable for producing deodorant products having excellent washing durability, such as deodorant products obtained by imparting a deodorizing effect to paper, woven fabrics, nonwoven fabrics, and polymer sheets or processed products thereof (e.g., clothes, interior accessories, nursing care products, medical products, filters, bags, shoes, building materials, and car interior materials).

1: A deodorant dispersion comprising:
(A) two or more powdery inorganic chemical adsorbents,
(B) a dispersing agent, and
(C) a dispersion medium,
wherein a content proportion of the component (A) is in a range from 10% to 30% by mass based on a total of the deodorant dispersion,
wherein a content proportion of the component (B) is in a range from 1% to 10% by mass based on a total of the deodorant dispersion, and
wherein a median particle diameter of a dispersoid is in a range from 0.1 μm to 0.4 μm.

2: The deodorant dispersion according to claim 1,
wherein a gas adsorption capacity of the component (A) is 25 mL/g or more.

3: The deodorant dispersion according to claim 1,
wherein at least one of the inorganic chemical adsorbents constituting the component (A) is an amorphous substance.

4: The deodorant dispersion according to claim 1,
wherein the component (A) comprises a compound represented by a formula (1):
aM¹₂O.bM²O.cM³₂O₃.dM⁴O₂.eP₂O₅.fH₂O (1)
wherein M¹ represents an alkali metal atom, M² represents a bivalent metal atom, M³ represents a trivalent metal atom, M⁴ represents a tetravalent metal atom, at least one of a, b, c, d and e is a positive number and the rest of a, b, c, d and e are each 0 or a positive number, and f is a positive number.

5: The deodorant dispersion according to claim 4,
wherein M¹₂O in the formula (1) is Na₂O.

6: The deodorant dispersion according to claim 4,
wherein M²O in the formula (1) is MgO, ZnO or CuO.

7: The deodorant dispersion according to claim 4,
wherein M³₂O₃ in the formula (1) is Al₂O₃.

8: The deodorant dispersion according to claim 4,
wherein M⁴O₂ in the general formula (1) is SiO₂, TiO₂ or ZrO₂.

9: A deodorant containing processing solution comprising the deodorant dispersion according to claim 1, and an adhesive agent.

10: A production method of a deodorant product, comprising:
a process for applying the deodorant containing processing solution according to claim 9 on a base material, and
a process for drying a coated article.
